(19) United States
(12) Patent Application Publication
(10) Pub. No.: US 2019/0369615 A1
(43) Pub. Date: Dec. 5, 2019

(54) SYSTEM AND METHOD FOR VERIFYING THE PRESENCE AND VITAL SIGNS OF PILOTS IN A COCKPIT OF AN AIRPLANE

(71) Applicant: Facultad Politecnica-Universidad Nacional del Este, Ciudad del Este (PY)

(72) Inventors: Eustaquio Alcides MARTINEZ JARA, Ciudad del Este (PY); Gabriela Matilde BOBADILLA DE ALMADA, Ciudad del Este (PY); Ruben Dario KANG CARDOZO, Ciudad del Este (PY); Osvaldo Miguel GONZALEZ PRIETO, Ciudad del Este (PY)

(21) Appl. No.: 16/030,588

(22) Filed: Jul. 9, 2018

(30) Foreign Application Priority Data
Jun. 5, 2018 (PY) P 1842725

(51) Int. Cl. G05D 1/00; G08G 5/00; G05D 1/02; B64C 13/18; A61B 5/00; A61B 5/024

(52) U.S. Cl. CPC G05D 1/0061; G08G 5/0095; G05D 1/0202; A61B 5/742; A61B 5/002; A61B 5/024; B64C 13/18

(57) ABSTRACT

The invention relates to a system and method for verifying the presence and vital signs of pilots in a cockpit of an airplane. The system uses autonomous short-range active communication equipment and, in particular, a central control and actuation unit located in the cockpit of the airplane and personal control units worn by the pilots that wait to receive a request signal from the central control and actuation unit to determine the absence of the pilot from the cockpit, based on the intensity of the request signal received, and whether the pilots' vital signs are anomalous, using a heart rate sensor. In the event of detecting an anomalous situation, an alert is sent to the central control and actuation unit, which executes a certain action in accordance with the specific alarm detected.

### CROSS REFERENCE TO RELATED APPLICATION

This application claims priority of Paraguay Application No. P 1842725 PY filed Jun. 5, 2018, application which is incorporated herein by reference.

### FIELD OF THE INVENTION

The present invention falls within the field of security systems and methods implemented in airplane cockpits.

### BACKGROUND OF THE INVENTION

Currently, as established in different aviation security regulations, aircraft must include onboard systems that make it possible to ensure the presence of authorised persons to fly the airplane.

With respect to the identification of the presence of persons in restricted spaces for security reasons, U.S. Pat. No. 9,041,546 discloses a system and a method for detecting the position of a person relative to a machine. However, said invention uses a passive transmitter and locator, and is limited to detecting presence in relation to a fixed transmitter that must be positioned on one or more machines, but is not aimed at detecting teams of people with freedom of movement inside the airplane.

U.S. Pat. No. 9,030,323 uses a method and device for detecting the position of an object using radio-frequency identification (RFID). The method includes calculating a position of a moving object using a first RFID tag installed at a particular position in a predetermined space while the moving object is moving, detecting a second RFID tag included in the object, determining the position of the same at a certain point of time.

A large number of inventions can be identified in the field of communication between electronic devices, but none addresses the problem of cockpit security, where the level of security must be increased to a maximum, without limiting said security to mere physical presence.

The present invention solves these problems, making it possible to ensure the active presence of pilots in cockpits.

### DESCRIPTION OF THE INVENTION

The invention relates to a system and a method that detects the active presence of human beings in a delimited space, specifically for cockpit security, using autonomous short-range active communication equipment.

The main objective of the invention is to increase the level of security of human beings who work in teams and must necessarily remain within a delimited area of the cockpit.

The invention makes it possible to ensure the presence, amount and vital signs of persons authorised to fly the plane. Said persons who have freedom of movement inside the plane are equipped with transmitters and actuators, working as a mobile distributed communication system, wherein each transmitter has an embedded system with decision-making capacity.

A first aspect of the present invention relates to a system for detecting the presence of pilots in the cockpit of an airplane, comprising: a central control and actuation unit located inside the cockpit of an airplane and which is equipped with a wireless communication module and data processing means; a plurality of heart rate sensors, each of which is adapted to be adhered to each pilot (fixed to the chest near the heart, to the wrist or to any part of the body where the heart rate can be easily detected) of the airplane to detect their vital signs; a plurality of personal control units, worn each of which is adapted to be by a pilot of the airplane and which comprises a wireless communication module, data processing means (e.g. a microcontroller), one or several batteries, and a heart rate sensor interface configured to receive a vital sign signal from a heart rate sensor once connected to the personal control unit.

Each personal control unit is configured to verify the receipt of a request signal sent by the central control and actuation unit; calculate the intensity of the request signal received; determine the absence of the pilot from the cockpit when said intensity falls below a certain threshold and, in such case, wirelessly send an absence alert message to the central control and actuation unit alerting of the absence of the pilot from the cockpit; determine, by analysing the vital sign signal received from the corresponding heart rate sensor, whether the pilot's heart rate is outside of a predetermined normal range, in which case an alert message alerting of the vital signs will be wirelessly sent to the central control and actuation unit alerting that the pilot's heart rate is outside of the normal range.

The central control and actuation unit is configured to wirelessly send a request signal to the personal control units; verify the receipt of alert messages from the personal control units; determine the existence of an alert situation in accordance with the alert messages received; and execute at least one action in response to the alert situation detected.

According to one embodiment, the central control and actuation unit is configured to determine the existence of an alert situation when it receives at least one vital sign alert message alerting that a pilot's heart rate is outside of the normal range and/or when it receives at least one absence alert message alerting of the absence of a pilot from the cockpit (for example, when it receives absence alert messages alerting that all the pilots are outside of the cockpit).

In one embodiment, the central control and actuation unit is configured, in response to a detected alert situation, to activate the autopilot, send an alarm message to a control tower and/or send an alarm message to the personal control units (which can, for example, produce an acoustic and/or light signal to alert the pilot.

Therefore, the system uses two types of devices, a central control and actuation unit and a personal control unit worn by each pilot, being both autonomous devices with processing capacity connected over a wireless network. The central control and actuation unit can be embedded in the Flight Management System (FMS) of the aircraft or can be an external device (a portable device encapsulated in a casing) located in the cockpit of the airplane. The central control and actuation unit is in charge of controlling, constantly recording and emitting alert signals alerting of the presence of the pilots and their state of health, while the personal control unit is worn by each member of the aircraft flying team.

With respect to the operation of the personal control unit worn by a pilot, the process begins by turning on said personal unit and waiting to receive a signal or code from a central control and actuation unit that is within its range. The receipt of the confirmation signal or code is verified; if affirmative, a predetermined time period is awaited and that established is executed; if negative, the logic established for that situation is also executed.

The use of the actuators makes it possible to increase the level of security in the work team. The actuators may block, interrupt or activate any device, in the event that the number of persons established in the control zone (i.e. the cockpit of an airplane) is not identified.

In accordance with one embodiment, the central control and actuation unit is an electronic device comprising a communication module, antenna, sensor interface, actuator interface, audiovisual indicator interface, backup memory, power source/battery, programming interface and a microcontroller with CPU functions. The communication module comprises all the types of short-range wireless data transmission that currently exist or will be proposed in the future. The antenna is a device suitable for the wireless data transmission technology specified in the communication module. The sensor interface corresponds to an electronic circuit capable of interacting with various sensor technologies applicable to the invention, such as those that detect objects, persons or other conditions associated with an active or inactive situation of a particular variable, such as keys, switches, etc. The audiovisual indicator interface corresponds to a set of communication ports that makes it possible to transmit an audiovisual signal or data to an external device consisting of a set of characters that may be exhibited by any information presentation technology, such as displays or other underlying technologies. The programming interface consists of a standard communication port compatible with current technologies and those that will be proposed in the future that will enable communication with a computational system wherefrom the algorithms to be executed by the CPU of the central control and actuation unit will be obtained. The backup memory is a module that stores the program that must be executed in the central control and actuation unit and the configuration data in accordance with the specific application of the system. This backup memory is non-volatile. The battery or power source supplies sufficient energy for the operation of the central control and actuation unit, with an autonomy in accordance with the application of the system and that can be recharged, in addition to its connection to the power grid in the event that it must be located in a predetermined fixed location.

The personal control unit consists, in accordance with one embodiment, of an electronic device made of a communication module, antenna, sensor interface, audiovisual indicator interface, backup memory, batteries, programming interface and a CPU microcontroller, having similar functions to those of the central control and actuation unit except in that relating to the power supply and to the actuation interface, which for this case is more limited in terms of power and number of ports.

A second aspect of the present invention relates to a method for detecting the presence of pilots in the cockpit of an airplane, comprising the following stages:
Equipping each pilot of the airplane with a personal control unit connected to a heart rate sensor.
Having a central control and actuation unit in the cockpit of an airplane.
Wireless remittance, from the central control and actuation unit, of a request signal to the personal control units.
Verification, by the personal control units, of the receipt of said request signal.
Calculation, by the personal control units, of the intensity of the request signal received.
Determination, by the personal control units, of the absence of the pilot from the cockpit when said intensity falls below a certain threshold and, in such case, wireless remittance of a first alert message to the central control and actuation unit alerting of the absence of the pilot from the cockpit.
Determination, by the personal control units, upon analysing the signal received from the corresponding heart rate sensor, of whether the pilot's heart rate is outside of a predetermined normal range and, in such case, wireless remittance of a second alert message to the central control and actuation unit alerting that the pilot's heart rate is outside of the normal range.
Verification, by the central control and actuation unit, of the receipt of alert messages sent by the personal control units.
Determination of the existence of an alert situation in accordance with the alert messages received.
Execution of at least one action in response to the alert situation detected.

### BRIEF DESCRIPTION OF THE FIGURES

What follows is a very brief description of a series of drawings that help to better understand the invention and which is presented as a non-limiting example thereof.

FIG. 1 shows a schematic view of the elements of the system of the present invention, central control and actuation unit and personal control units, applied to the pilots and to a cockpit of an aircraft.

FIG. 2 shows the block diagram of a personal control unit that forms part of the system.

FIG. 3 shows the block diagram of a central control and actuation unit that forms part of the system.

FIG. 4 shows the flow diagram of the operating logic of the central control and actuation unit.

FIG. 5 shows the flow diagram of the operating logic of a personal control unit.

### DETAILED DESCRIPTION OF THE INVENTION

The present invention relates to a system and method that detects the presence and vital signs of human beings in a delimited space, such as the cockpit of an airplane, acting in accordance with the verification made.

The system comprises a central control and actuation unit and a personal control unit worn by each pilot or person authorised to fly the airplane. Both devices are connected by wireless communication.

The central control and actuation unit may be autonomous or adapted to a machine whereon it can act based on the established algorithm, taking as input variables the amount of personal control units detected within the established radius.

The personal control unit is a necessarily mobile device with unique identification that must be worn by each human being who forms part of the work team and has an energy storage device (for example, one or more batteries).

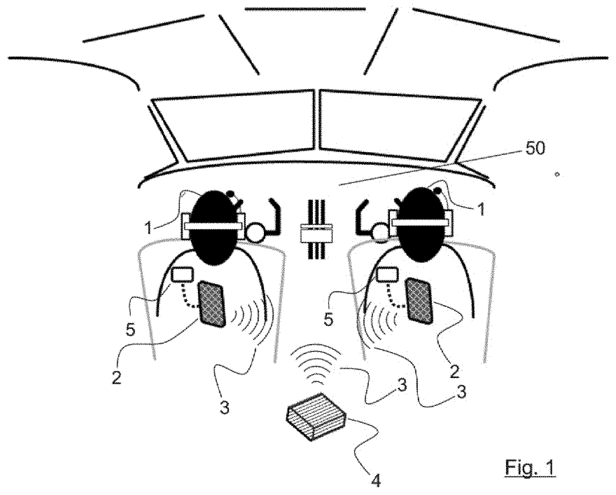

FIG. 1 shows an operating diagram of the invention, including the central control and actuation unit 4 and the personal control units 2, in addition to the possible installation in the cockpit 50 of an airplane and the pilots 1 or persons wearing the personal control units 2.

The system makes it possible to maximise security in airplanes, since the presence of the pilots in the cockpit is an essential factor. The personal control units 2 send, via a wireless signal 3, data to the central control and actuation unit 4, which analyses the signals received and decision-making based on the data contained in said signals, in accordance with predetermined internal programming. Additionally, the personal control units are equipped with a heart rate sensor 5 that is adhered to the pilot 1.

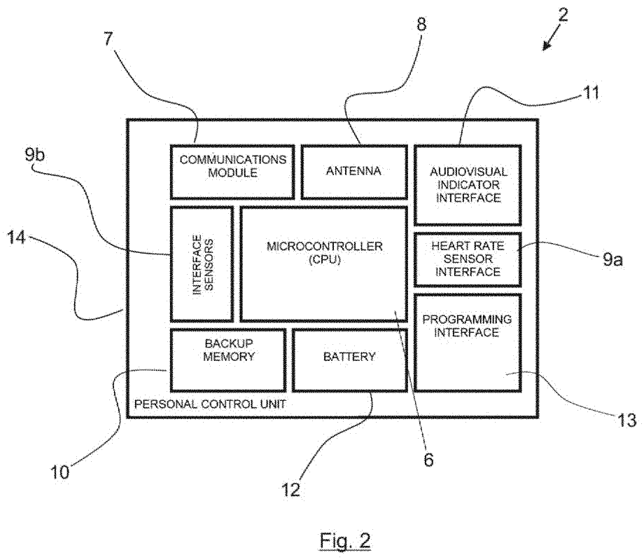

FIG. 2 shows a block diagram of the elements that form part of the personal control unit 2, in accordance with a possible embodiment. The personal control unit 2 is a device having computational processing capacity, a task performed by a microcontroller 6, which corresponds to the central processing unit or CPU. This microcontroller 6 receives and sends data through a wireless communication module 7, using an antenna 8. In addition to communication capacity, the personal control unit 2 receives the status of the heart rate sensor 5 via a heart rate sensor interface 9a, and can also receive statuses of different types of sensors through a sensor interface 9b. The configuration of the device, in addition to the programme it runs, is stored in a backup memory 10. Depending on the specific programming and application, if necessary, this device can emit audiovisual signals through an audiovisual indicator interface 11, using acoustic or light devices or eventually a display. Since the device is electronic, it requires a power source, a function fulfilled by a rechargeable battery 12. The programming of the device and the specification of parameters such as unique identification or ID are established through a programming interface 13, which can be a standard communication port in accordance with the available technology. The personal control unit 2 is encapsulated in a casing 14.

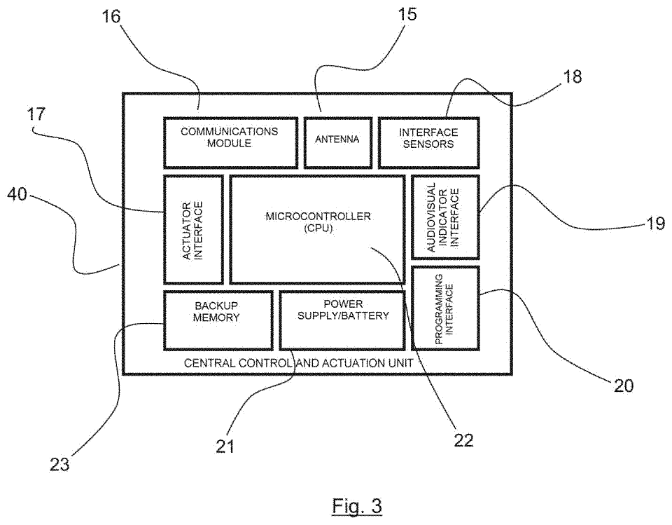

FIG. 3 shows a block diagram of the central control and actuation unit 4, in accordance with a possible embodiment. This device has an antenna 15 and a wireless communication module 16 through which it interacts with the nearby personal control units 2, detected by means of a unique identification (ID) thereof. The central control and actuation unit 4 also has an interface for actuators 17, which makes it possible to perform the actions of turning on, turning off, blocking and unblocking electric or electronic devices that can act to activate and deactivate security elements of a wide variety of types and technologies. The central control and actuation unit 4 can also receive signals from various types of sensors through a sensor interface 18, depending on the application, to aid decision-making and enable the corresponding actuators. As in the case of the personal control unit 2, the central control and actuation unit 4 has an audiovisual indicator interface 19 that makes it possible to interact with sound-emitting devices, a light signal and eventually or other technology available for this purpose, such as a display. The programming, data loading and specific parameters, such as unique identification, is performed through a programming interface 20 and, since the central control and actuation unit 4 is an electronic device, it requires a power source 21 or a battery. The data processing of the portable device represented in FIG. 1 is executed by a microcontroller 22, with the help of the backup memory 23. The central control and actuation unit 4 is encapsulated in a casing 40. Alternatively, instead of a portable device, the central control and actuation unit 4 can be included in the electronics of the aircraft itself, for example, it can form part of the Flight Management System (FMS).

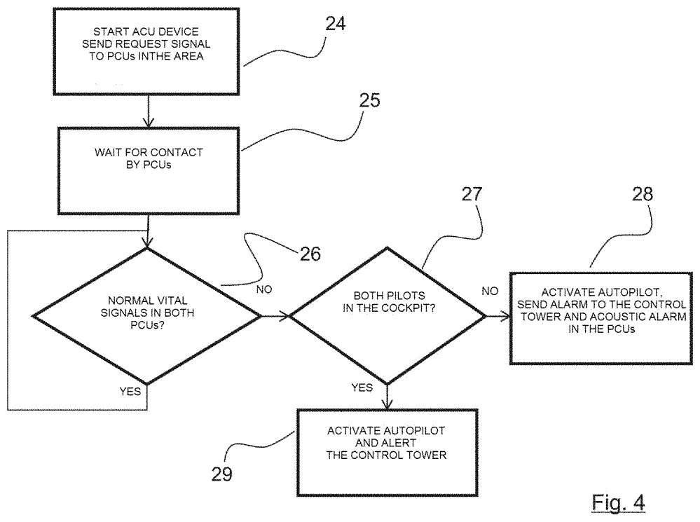

FIG. 4 represents a flow diagram of the operating logic of the central control and actuation unit 4. This device sends 24 a massive request signal (broadcast) aimed at all the personal control units 2 previously recorded in its memory. Next, it waits 25 for the responses from the personal control units 2, which respond with a message, including its unique code or identifier (ID), with an alert signal if the pilot's heart rate is outside of the normal range and an alert signal if the pilot is outside of the cockpit. The central control and actuation unit 4 verifies 26 whether the heart rate sensors 5 of all the personal control units 2 display normal vital sign readings; in the event that some of the vital sign readings are not normal, it will be verified 27 whether the two pilots are in the cockpit 27. In the event that either of them are outside of the cockpit 28, the autopilot is activated, it sends an alarm to the control tower and an acoustic alarm is emitted in the personal control units 2. If both pilots are in the cockpit when the reading of one of the sensors is abnormal, the autopilot is activated 29 and an alarm is sent to the control tower. It should be noted that the specific actions performed in steps 28 and 29 may vary in different embodiments, since what is important about this process is the combined monitoring of the presence of the pilots in the cockpit and of their vital signs, acting adequately in accordance with a predetermined programming as soon as an abnormal situation of these parameters is detected.

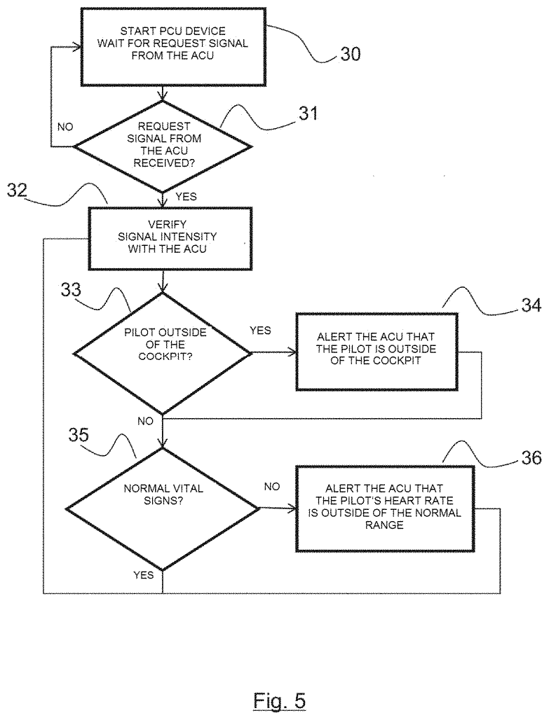

FIG. 5 shows a flow diagram of the operating logic of the personal control unit 2, which is the device worn by the pilots 1. The process is initiated by turning on 30 the personal control unit 2, which awaits the receipt of a request signal from the central control and actuation unit 4, when it is within the range thereof. The receipt of said request signal is verified 31 and, in the event that no request signal is received, it waits for a certain time period. When the personal control unit 2 receives said request signal, it calculates 32 the intensity of the request signal previously received from the central control and actuation unit 4 to determine 33 whether the pilot 1 is present in the cockpit 50; in the event of receiving a request signal below a certain threshold, the personal control unit 2 determines that the pilot 1 is outside of the cockpit 50 and, in the event of receiving a strong request signal (above a threshold), it determines that the pilot 1 is inside the cockpit 50. Upon confirming that the pilot is outside of the cockpit, the personal control unit 2 sends 34 a first alert message to the central control and actuation unit 4 alerting of this situation. Next, the personal control unit 2 verifies 35 that the pilot's heart rate is within the predetermined normal value. If not, the personal control unit 2 sends 36 a second alert message, a vital sign alert message, to the central control and actuation unit 4 alerting that the heart rate is outside of the normal range. Next, the cycle is repeated once again.

1. A system for verifying the presence and vital signs of pilots in a cockpit of an airplane, comprising:
a central control and actuation unit located in the cockpit of an airplane and equipped with a wireless communication module and data processing means;
a plurality of heart rate sensors, wherein each heart rate sensor is adapted to be adhered to each pilot of the airplane to detect their vital signs;
a plurality of personal control units, wherein each personal control unit is adapted to be worn by a pilot of the airplane and comprises a wireless communication module, data processing means, at least one battery and a heart rate sensor interface configured to receive a vital sign signal from a heart rate sensor once connected to the personal control unit;
wherein each personal control unit is configured to:
verify the receipt of a request signal sent by the central control and actuation unit;
calculate the intensity of the request signal received;
determine the absence of the pilot from the cockpit when said intensity falls below a certain threshold and, in such case, wirelessly send an absence alert message to the central control and actuation unit alerting of the absence of the pilot from the cockpit;
determine, by analysing the vital sign signal received from the corresponding heart rate sensor, whether the pilot's heart rate is outside of a predetermined normal range and, in such case, wirelessly send a vital sign alert message to the central control and actuation unit alerting that the pilot's heart rate is outside of the normal range;
wherein the central control and actuation unit is configured to:
wirelessly send a request signal to the personal control units;
verify the receipt of alert messages from the personal control units;
determine the existence of an alert situation in accordance with the alert messages received;
execute at least one action in response to the alert situation detected.

2. The system of claim 1, wherein the central control and actuation unit is a portable device encapsulated in a casing.

3. The system of claim 1, wherein the central control and actuation unit is a device embedded in the flight management system of the airplane.

4. The system, according to claim 1, wherein the central control and actuation unit is configured to determine the existence of an alert situation when it receives at least one vital sign alert message alerting that the heart rate of a pilot is outside of the normal range.

5. The system, according to claim 1, wherein the central control and actuation unit is configured to determine the existence of an alert situation when it receives at least one absence alert message alerting of the absence of a pilot from the cockpit.

6. The system, according to claim 1, wherein the central control and actuation unit is configured to activate the autopilot in response to an alert situation detected.

7. The system, according to claim 1, wherein the central control and actuation unit is configured to send an alarm message to a control tower in response to an alert situation detected.

8. The system, according to claim 1, wherein the central control and actuation unit is configured to send an alarm message to the personal control units in response to an alert situation detected.

9. A method for verifying the presence and vital signs of pilots in a cockpit of an airplane, comprising:
equipping each pilot of the airplane with a personal control unit connected to a heart rate sensor;
having a central control and actuation unit in the cockpit of an airplane;
wirelessly sending, from the central control and actuation unit, a request signal to the personal control units;
verifying, by the personal control units, the receipt of said request signal;
calculating, by the personal control units, the intensity of the request signal received;
determining, by the personal control units, the absence of the pilot from the cockpit when said intensity falls below a certain threshold and, in such case, wireless remittance of a first alert message to the central control and actuation unit alerting of the absence of the pilot from the cockpit;
determining, by the personal control units, through analysis of the signal received from the corresponding heart rate sensor, of whether the pilot's heart rate is outside of a predetermined normal range and, in such case, wireless remittance of a second alert message to the central control and actuation unit alerting that the heart rate of the pilot is outside of the normal range;
verifying, by the central control and actuation unit, the receipt of alert messages from the personal control units;
determining the existence of an alert situation in accordance with the alert messages received;
executing at least one action in response to the alert situation detected.

10. The method, according to claim 9, wherein the existence of an alert situation is determined when the central control and actuation unit receives at least one vital sign alert message alerting that the heart rate of a pilot is outside of the normal range.

11. The method, according to claim 9, wherein the existence of an alert situation is determined when the central control and actuation unit receives at least one absence alert message alerting of the absence of a pilot from the cockpit.

12. The method, according to claim 9, comprising activating the autopilot in response to an alert situation detected.

13. The method, according to claim 9, comprising sending an alarm message to a control tower in response to an alert situation detected.

14. The method, according to claim 9, comprising sending an alarm message to the personal control units in response to an alert situation detected.
